(19) United States
(12) Patent Application Publication
(10) Pub. No.: US 2023/0172825 A1
(43) Pub. Date: Jun. 8, 2023

(54) COMPOSITION FOR IMPROVING SKIN COMPRISING AMINO ACID COMPLEX AS ACTIVE INGREDIENT

(71) Applicants: NATURE IN LAB. INC., Naju-si (KR); BEAUTY & K CO., LTD., Seoul (KR)

(72) Inventors: Jaeheon MOON, Anyang-si, Gyeonggi-do (KR); You Ri KO, Seoul (KR)

(73) Assignees: NATURE IN LAB. INC., Naju-si (KR); BEAUTY & K CO., LTD., Seoul (KR)

(21) Appl. No.: 18/073,752

(22) Filed: Dec. 2, 2022

(30) Foreign Application Priority Data
Dec. 3, 2021 (KR) 10-2021-0172234

(51) Int. Cl. A61K 8/44; A61K 8/19; A61Q 19/08

(52) U.S. Cl. CPC A61K 8/44; A61K 8/19; A61Q 19/08

(57) ABSTRACT

Provided is a composition for skin improvement, including an amino acid complex as an active ingredient. The composition according to an aspect including the amino acid complex as an active ingredient may proliferate cells, promote expression and synthesis of collagen, and have an effect on skin wrinkles and skin elasticity, and thus may be effectively used for overall skin improvements.

### CROSS-REFERENCE TO RELATED APPLICATION

This application is based on and claims priority under 35 U.S.C. §119 to Korean Patent Application No. 10-2021-0172234, filed on Dec. 03, 2021, in the Korean Intellectual Property Office, the disclosure of which is incorporated by reference herein in its entirety.

### BACKGROUND

### 1. Field

One or more embodiments relate to a composition including an amino acid complex as an active ingredient for skim improvement.

### 2. Description of the Related Art

Skin aging is a natural phenomenon that progresses over time, and is not a property that can be fundamentally prevented although there are differences among individuals as in the aging of the human body. However, causes of skin aging include not only intrinsic factors that progress over time, but also extrinsic factors, such as smoking, excessive alcohol drinking, undernutrition, chronic exposure to sunlight, and the like, that can be prevented. Accordingly, it is known that skin aging and skin wrinkles derived therefrom cannot be prevented for life, but can be delayed. Attempts to slow down aging of the skin through cosmetics have been studied a lot in the cosmetic field, and this field is still the most interested and researched field.

The skin undergoes various changes through aging. First, the thicknesses of components of the skin, i.e., epidermis, dermis, and hypodermis, may be reduced, and extracellular matrix (ECM) components giving elasticity to the skin may change. Among the ECM components, production of collagen, which accounts for about 70 % to about 80 % of the ECM, is rapidly decreased, and in this regard, collagen may have a close relationship with production of wrinkles. Collagen, elastin, proteoglycan, glucosaminoglycan, laminin, fibronectin, and the like, which constitute the skin connective tissues, lose functions thereof by oxidation, and thus the skin loses elasticity and wrinkles are excessively formed. Accordingly, the skin is changed into senile skin.

Fibers in connective tissue of the ECM include collagen fibers, reticular fibers, and elastic fibers, and collagen which accounts for about 70 % of the skin connective tissue is mostly formed in fibroblasts of the skin. The amount of collagen within the skin connective tissue decreases with age, and such a decrease is due to a decrease in collagen synthesis and promotion of collagen decomposition. Therefore, the decrease in collagen biosynthesis and the promotion of collagen decomposition are the biggest causes of the formation of skin wrinkles.

In addition, skin wrinkles are also related to skin moisturizing. This is because that, when the skin is not moisturized well, skin damage occurs as the skin becomes dry and rough, and accordingly, wrinkles are easily formed depending on the expression of the face.

About 70 % of body weight of an adult is water, and the skin not only prevents invasion of viruses or bacteria, but also suppresses evaporation of moisture. Substances that play an important role in moisturizing of the skin may include a natural moisturizing factor (NMF) in the stratum corneum of the skin. 40 % of the NMF may consist of amino acids.

In addition, collagen, which is related to the skin elasticity and causes the formation of wrinkles as described above, may also consist of amino acids, and various factors related to the skin barrier, such as filaggrin, loricrin, and the like, may also consist of amino acids.

Accordingly, the present inventors have confirmed an effect of a composition, which includes amino acids that are the most basic and essential, on the skin improvement, thereby completing the present disclosure.

### SUMMARY

An aspect is to provide a cosmetic composition for skin improvement, including, as an active ingredient, an amino acid complex including glycine, L-alanine, L-proline, L-valine, L-leucine, L-lysine, serine, glutamic acid, aspartic acid, arginine, and histidine.

Another aspect is to provide a food composition for skin improvement, including the amino acid complex as an active ingredient.

Additional aspects will be set forth in part in the description which follows and, in part, will be apparent from the description, or may be learned by practice of the presented embodiments of the disclosure.

An aspect provides a cosmetic composition for skin improvement, including, as an active ingredient, an amino acid complex including glycine, L-alanine, L-proline, L-valine, L-leucine, L-lysine, serine, glutamic acid, aspartic acid, arginine, and histidine.

These amino acids may each independently be an L-type amino acid or a D-type amino acid. Amino acids, such as alanine, proline, valine, leucine, and lysine, that can have an L-form are represented by L-amino acids, but the form of L-amino acids may be converted to a D-form depending on a treatment environment. Thus, the form of amino acids is not limited by the representation above.

In an embodiment, the composition may further include, as active ingredients, calcium pantothenate and a mineral component.

The calcium pantothenate is a calcium salt of water-soluble vitamin B5.

In an embodiment, the mineral component may include at least three selected from the group consisting of calcium (Ca), magnesium (Mg), copper (Cu), and zinc (Zn), and may be in the form of a single substance or a salt. The mineral component may include at least three minerals selected from the group consisting of Zn that plays a role in cell regeneration, Mg that plays a role in maintaining skin balance, Cu that plays a role in synthesis of proteins and collagen, and Ca that is more effective in antiaging by promoting epidermal differentiation, in the form of a single substance or a salt. In addition, the mineral in the form of a salt may include aspartate or gluconate of the mineral component. For example, the mineral component may include at least three minerals selected from the group consisting of Mg aspartate, Cu gluconate, Zn gluconate, Ca gluconate, and Mg gluconate.

The mineral component may include at least three selected from the group consisting of Ca, Mg, Cu, and Zn at a weight ratio of 1 to 3:1 to 3:1 to 3 or 1:1:1, or at a weight ratio of 1 to 3:1 to 3:1 to 3: 1 to 3 or 1:1:1:1.

In an embodiment, the composition may include, based on the total weight of the amino acid complex, glycine in an amount of about 20.0 wt% to about 35.0 wt%, L-alanine in an amount of about 15.0 wt% to about 30.0 wt%, L-proline in an amount of about 15.0 wt% to about 20.0 wt%, L-valine in an amount of about 10.0 wt% to about 25.0 wt%, L-leucine in an amount of about 1.0 wt% to about 5.0 wt%, L-lysine in an amount of about 1.0 wt% to about 5.0 wt%, serine in an amount of about 5.0 wt% to about 10.0 wt%, glutamic acid in an amount of about 5.0 wt% to about 10.0 wt%, aspartic acid in an amount of about 1.0 wt% to about 5.0 wt%, arginine in an amount of about 1.0 wt% to about 5.0 wt%, and histidine in an amount of about 1.0 wt% to about 5.0 wt%.

For example, the composition may include, based on the total weight of the amino acid complex, glycine in an amount of about 20.0 wt% to about 35.0 wt%, about 20.0 wt% to about 32.0 wt%, about 20.0 wt% to about 30.0 wt%, about 20.0 wt% to about 28.0 wt%, about 22.0 wt% to about 35.0 wt%, about 22.0 wt% to about 32.0 wt%, or about 22.0 wt% to about 30.0 wt%, L-alanine in an amount of about 15.0 wt% to about 30.0 wt%, about 15.0 wt% to about 28.0 wt%, about 15.0 wt% to about 25.0 wt%, about 15.0 wt% to about 23.0 wt%, or about 15.0 wt% to about 20.0 wt%, L-proline in an amount of about 15.0 wt% to about 20.0 wt%, about 16.0 wt% to about 20.0 wt%, or about 18.0 wt% to about 20.0 wt%, L-valine in an amount of about 10.0 wt% to about 25.0 wt%, about 10.0 wt% to about 23.0 wt%, about 10.0 wt% to about 21.0 wt%, about 10.0 wt% to about 20.0 wt%, or about 10.0 wt% to about 18.0 wt%, L-leucine in an amount of about 1.0 wt% to about 5.0 wt% or about 2.0 wt% to about 5.0 wt%, L-lysine in an amount of about 1.0 wt% to about 5.0 wt% or about 2.0 wt% to about 5.0 wt%, serine in an amount of about 5.0 wt% to about 10.0 wt% or about 5.0 wt% to about 8.0 wt%, glutamic acid in an amount of about 5.0 wt% to about 10.0 wt% or about 5.0 wt% to about 8.0 wt%, aspartic acid in an amount of about 1.0 wt% to about 5.0 wt% or about 2.0 wt% to about 4.0 wt%, arginine in an amount of about 1.0 wt% to about 5.0 wt% or about 2.0 wt% to about 5.0 wt%, and histidine in an amount of about 1.0 wt% to about 5.0 wt% or about 1.0 wt% to about 3.0 wt%.

In an embodiment, the composition may include, based on the total weight of the composition, the amino acid complex in an amount of about 10.0 wt% to about 40.0 wt%. For example, the composition may include, based on the total weight of the composition, the amino acid complex in an amount of about 10.0 wt% to about 40.0 wt%, about 10.0 wt% to about 35.0 wt%, about 10.0 wt% to about 30.0 wt%, about 10.0 wt% to about 25.0 wt%, about 15.0 wt% to about 40.0 wt%, about 15.0 wt% to about 35.0 wt%, about 15.0 wt% to about 30.0 wt%, about 15.0 wt% to about 25.0 wt%, about 20.0 wt% to about 40.0 wt%, about 20.0 wt% to about 35.0 wt%, or about 20.0 wt% to about 30.0 wt%.

In an embodiment, the composition may include, based on the total weight of the composition, the calcium pantothenate in an amount of about 0.1 wt% to about 5.0 wt%. For example, the composition may include, based on the total weight of the composition, the calcium pantothenate in an amount of about 0.1 wt% to about 5.0 wt%, about 0.1 wt% to about 3.0 wt%, about 0.1 wt% to about 1.0 wt%, about 0.5 wt% to about 5.0 wt%, about 0.5 wt% to about 3.0 wt%, or about 0.5 wt% to about 1.0 wt%.

In an embodiment, the composition may include, based on the total weight of the composition, the mineral component in an amount of about 0.1 wt% to about 5.0 wt%. For example, the composition may include, based on the total weight of the composition, the mineral component in an amount of about 0.1 wt% to about 5.0 wt%, about 0.1 wt% to about 3.0 wt%, about 0.1 wt% to about 1.0 wt%, about 0.5 wt% to about 5.0 wt%, about 0.5 wt% to about 3.0 wt%, or about 0.5 wt% to about 1.0 wt%.

The skin improvement may be at least one selected from the group consisting of improvement of skin barrier, prevention or improvement of skin damage, skin moisturizing, amelioration of skin wrinkles, inhibition of skin aging, improvement of skin elasticity, skin soothing, and skin regeneration.

The present inventors confirmed that the composition including the amino acid complex proliferates cells, promotes expression and synthesis of collagen, and has an effect on skin wrinkles and skin elasticity. Accordingly, the composition according to an embodiment may exhibit effects on overall skin improvements related to improvement of skin barrier, prevention or improvement of skin damage, skin moisturizing, amelioration of skin wrinkles, improvement of skin elasticity, skin soothing, and skin regeneration.

The term “skin damage” refers to an external stimulus, for example, death of human skin cells by ultraviolet rays, DNA damage of skin cells, increased reactive oxygen species, increased lipid peroxidation, and the like, and symptoms of the skin damage may include erythema, sunburn, pigmentation, photoaging, skin cancer, and the like.

The cosmetic composition may be prepared in a formulation including a face lotion (skin lotion), a face toner, a skin softener, a skin toner, an astringent, a lotion, a milky lotion, a moisturizing lotion, a nourishing lotion, a massage cream, a nourishing cream, a moisturizing cream, a hand cream, a hand sanitizer, a foundation, an essence, a nutrient essence, a cosmetic pack, a soap, a cleansing foam, a cleansing lotion, a cleansing cream, a body lotion, a body cleanser, a suspension, a gel type, a powder type, a paste, a mask pack, and a mask sheet. The cosmetic composition of such a formulation may be prepared according to a method known in the art. A blending amount of an additional ingredient such as a moisturizer or the like may be easily selected by those skilled in the art within a range that does not impair the purpose and effect of the present disclosure.

The cosmetic composition may additionally include a functional additive and a component included in general cosmetic compositions, in addition to the active ingredients disclosed herein, and may further include commonly used substances, such as purified water, a thickener, a preservative, a stabilizer, a solubilizer, a surfactant, a carrier, a fragrant substance, or a combination thereof. The functional additive may include a component selected from the group consisting of a water-soluble vitamin, an oil-soluble vitamin, a polymer peptide, a polymer polysaccharide, a sphingolipid, and a seaweed extract. Examples of the carrier include alcohol, oil, a surfactant, fatty acid, silicone oil, a wetting agent, a moisturizing agent, a viscosity modifier, an emulsion, a stabilizer, a UV scattering agent, a UV absorber, a color developer, a fragrant substance, and the like. Compounds/compositions that can be used as the alcohol, oil, surfactant, fatty acid, silicone oil, wetting agent, moisturizing agent, viscosity modifier, emulsion, stabilizer, UV scattering agent, UV absorber, color developer, and fragrant substance are already known in the art, and thus those skilled in the art may select and use appropriate materials/compositions. In addition, the cosmetic composition may further include, as necessary, a sunscreen agent, an antioxidant (e.g., butylhydroxyanisole, propyl gallate, erythorbic acid, tocopheryl acetate, butylated hydroxytoluene, etc.), a preservative (e.g., methylparaben, butylparaben, propylparaben, phenoxyethanol, imidazolidinyl urea, chlorphenesin, etc.), a colorant, a pH adjuster (e.g., triethanolamine, citric acid, citrate, sodium citrate, malic acid, sodium malate, fumaric acid, sodium fumarate, succinic acid, sodium succinate, sodium hydroxide, dibasic sodium phosphate, etc.), a moisturizing agent (e.g., glycerin, sorbitol, propylene glycol, butylene glycol, hexylene glycol, diglycerin, betaine, glycerethe-26, methyl gluceth-20, etc.), a lubricant, and the like.

In addition, in the cosmetic composition of each formulation, appropriate components may be selected and blended according to a formulation type or a purpose of use of cosmetics. The components and methods for blending may follow the techniques known in the art, and thus a detailed description thereof will be omitted in the present specification.

Another aspect provides a food composition for skin improvement, including, as an active ingredient, an amino acid complex including glycine, L-alanine, L-proline, L-valine, L-leucine, L-lysine, serine, glutamic acid, aspartic acid, arginine, and histidine.

In an embodiment, the composition may further include, as active ingredients, calcium pantothenate and a mineral component.

The amino acids, calcium pantothenate, and mineral component, and the effect of the composition on the skin improvement are the same as described above.

When the amino acid complex, calcium pantothenate, and mineral component are used as food additives, these active ingredients may be added intactly or used in combination with other foods or food ingredients, and may be appropriately used according to a method known in the art. A mixing amount of the active ingredients may be appropriately determined according to the purpose of use (e.g., prevention, health care, or therapeutic treatment). For example, the composition may include, based on the total weight of the composition, the amino acid complex in an amount of about 0.0001 wt% to about 99.0 wt%, for example, about 0.01 wt% to about 60 wt%, about 0.01 wt% to about 40 wt%, about 0.01 wt% to about 30 wt%, about 0.01 wt% to about 20 wt%, about 0.01 wt% to about 10 wt%, about 0.01 wt% to about 5 wt%, about 0.05 wt% to about 60 wt%, about 0.05 wt% to about 40 wt%, about 0.05 wt% to about 30 wt%, about 0.05 wt% to about 20 wt%, about 0.05 wt% to about 10 wt%, about 0.05 wt% to about 5 wt%, about 0.1 wt% to about 60 wt%, about 0.1 wt% to about 40 wt%, about 0.1 wt% to about 30 wt%, about 0.1 wt% to about 20 wt%, about 0.1 wt% to about 10 wt%, about 0.1 wt% to about 5 wt%, about 5 wt% to about 20 wt%, about 5 wt% to about 18 wt%, about 6 wt% to about 15 wt%, about 8 wt% to about 15 wt%, or about 7 wt% to about 10 wt%. However, in the case of long-term intake for health and hygiene purposes or for health control purposes, the amount of the amino acid complex may be less or equal to the ranges above. Since there is no problem related to safety, the active ingredients may be used in an amount greater than or equal to the ranges above.

Types of the food are not particularly limited. Examples of the food to which the substances can be added include formulations selected from the group consisting of a powder, a granule, a tablet, a capsule, a pill, a gel, a jelly, a suspension, an emulsion, a syrup, a tea bag, a leached tea, and a health beverage. The food may include all types of health food in the ordinary sense.

A composition for the health beverage may include, as additional ingredients, various flavoring agents or natural carbohydrate, as in cases of conventional beverages. Examples of the natural carbohydrate include: a monosaccharide such as glucose and fructose; and a disaccharide such as maltose and sucrose, and a natural sweetening agent, such as dextrin and cyclodextrin, and a synthetic sweetening agent, such as saccharin and aspartame, may be used. Here, a proportion of the natural carbohydrate may generally be in a range of about 0.01 g to about 10 g, preferably, about 0.01 g to about 0.1 g, per 100 ml of the composition of the present disclosure.

The food may include a sitologically acceptable food supplement additive. For example, a suitable carrier that is commonly used in preparation of the food may be used.

In addition to the substances described above, the composition of the present disclosure may include various nutrients, vitamins, electrolytes, flavoring agents, colorants, pectic acid and a salt thereof, alginic acid and a salt thereof, organic acids, protective colloidal thickeners, pH adjusters, stabilizers, preservatives, glycerin, alcohol, carbonizing agents used in soft drink, and the like. In addition, the composition of the present disclosure may include fruit pulps for preparing a natural fruit juice, a fruit drink, and a vegetable drink. These components may be used independently or in combination with other components. Here, a ratio of the additives is not so important, but may be generally selected within a range of about 0.1 part by weight to about 20 parts by weight based on 100 parts by weight of the composition of the present disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a graph confirming cytotoxicity of a composition according to an embodiment;

FIG. 2 is a graph showing an effect of a composition according to an embodiment on mRNA expression of collagen type I;

FIG. 3 is a graph showing an effect of a composition according to an embodiment on mRNA expression of collagen type III; and

FIG. 4 is a graph showing an effect of a composition according to an embodiment on inhibition of MMP-1 in cells damaged by ultraviolet rays.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail through Examples below. However, these Examples are for illustrative purposes of one or more embodiments, and the scope of the present disclosure is not limited thereto.

### Examples and Comparative Examples: Preparation of Amino Acid Complex

For the amino acid complex included in the composition of the present disclosure, amino acids shown in Table 1 were included to prepare each amino acid complex. Here, the amount was represented by wt%.

Amino acid complexes D to F were each prepared by excluding some natural amino acids.

TABLE 1

| Amino acid complex | A | B | C | D | E | F |
|---|---|---|---|---|---|---|
| Glycine | 24.2 | 26.1 | 26.3 | 31.6 | 28.1 | 25.9 |
| L-Alanine | 18.2 | 19.5 | 19.7 | - | 26.0 | 19.5 |
| L-Proline | 18.2 | 19.5 | 19.5 | 19.8 | - | 19.5 |
| L-Valine | 13.4 | 15.1 | 16.1 | 21.0 | 18.5 | 15.1 |
| L-Leucine | 3.4 | 4.0 | 4.0 | 5.0 | 4.8 | - |
| L-Lysine | 2.6 | 3.7 | 3.7 | 2.6 | 2.6 | - |
| Serine | 6.2 | - | 6.2 | 6.2 | 6.2 | 6.2 |
| Glutamic acid | 5.7 | 5.7 | - | 5.7 | 5.7 | 5.7 |
| Aspartic acid | 3.6 | 3.6 | - | 3.6 | 3.6 | 3.6 |
| Arginine | 2.8 | 2.8 | 2.8 | 2.8 | 2.8 | 2.8 |
| Histidine | 1.7 | - | 1.7 | 1.7 | 1.7 | 1.7 |
| Sum | 100.0 | 100.0 | 100.0 | 100.0 | 100.0 | 100.0 |

Compositions each including the amino acid complex prepared above, calcium pantothenate, and a mineral component were prepared as shown in Table 2.

TABLE 2

| | Calcium pantothenate (wt%) | Mineral component^{a}(wt%) | Amino acid complex (wt%) |
|---|---|---|---|
| Example 1 | 0.7 | 0.6 | A : 25.0 |
| Example 2 | 0.7 | 0.6 | B : 25.0 |
| Example 3 | 0.7 | 0.6 | C : 25.0 |
| Comparative Example 1 | 0.7 | 0.6 | D : 25.0 |
| Comparative Example 2 | 0.7 | 0.6 | E : 25.0 |
| Comparative Example 3 | 0.7 | 0.6 | F : 25.0 |
| Comparative Example 4 | 0.7 | 0.6 | A : 9.0 |
| Comparative Example 5 | 0.7 | 0.6 | - |
| Comparative Example 6 | 0.7 | - | A : 25.0 |

Mineral component^{a}: Complex of Zn-gluconate, Mg-gluconate, and Ca-gluconate at a weight ratio of 1:1:1

### Experimental Example 1: Confirmation of Skin Cell Proliferation Effect

The effect of the composition including the amino acid complex, the calcium pantothenic acid salt, and the mineral component on proliferation of skin cells was confirmed.

In detail, human normal fibroblasts (available from Korean Cell Line Bank) were inoculated into a 96-well microplate to have a concentration of 1×10³ cells in each well, and cultured in DMEM (manufactured by Sigma company) 37° C. for 24 hours. Subsequently, the cells of experimental groups in which the prepared composition was included and a medium was replaced with fresh serum-free DMEM were further cultured for 36 hours, whereas the cells of a control group were further cultured for 36 hours without medium replacement. Next, 10 µl of MTT solution (5 mg/ml, 3-(4,5-dimethylthiazole-2-yl)2,5-diphenyltetrazolium bromide) was added to each well, and the cells were cultured for 6 hours. After removing the medium, 100 µl of DMSO solution was added to each well, and the cells were cultured by shaking for 20 minutes. Then, by using a microplate reader (Molecular Devices, USA), the absorbance of the supernatant was measured at 570 nm. The cell proliferation effect was calculated by substituting the measured value into Equation 1, and the results are shown in Table 3:

$$\text{Cell proliferation effect} \left(\%\right) =$$

$$\frac{A b s o r b a n c e o f e x p e r i m e n t a l g r o u p - A b s o r b a n c e o f c o n t r o l g r o u p}{A b s o r b a n c e o f c o n t r o l g r o u p} \text{x 100}$$

TABLE 3

| Entry | Cell proliferation effect (%) |
|---|---|
| Example 1 | 88.3 |
| Example 2 | 81.2 |
| Example 3 | 80.4 |
| Comparative Example | 67.9 |
| Comparative Example 2 | 68.4 |
| Comparative Example 3 | 69.2 |
| Comparative Example 4 | 53.1 |
| Comparative Example 5 | 33.4 |
| Comparative Example 6 | 51.2 |

As shown in Table 3, it was found that the composition including the amino acid complex exhibited a significantly high effect on the proliferation of fibroblasts. In addition, it was confirmed that, as in cases of Examples 1 to 3, the compositions that essentially included glycine, L-alanine, L-proline, L-valine, L-leucine, and L-lysine exhibited better effects, whereas Comparative Examples 1 to 3 in which some of these amino acids were not included exhibited somewhat less effects. Meanwhile, as in case of Comparative Example 4, the effect was reduced when the amount of the amino acid complex was low. In addition, in case of Comparative Example 5 in which the amino acid complex was not included, the effect on the cell proliferation appeared to be low, and in case of Comparative Example 6 in which the amino acid complex was not used together with the mineral component, the effect on the cell proliferation was also low.

### Experimental Example 2: Confirmation of Enhancement Effect on Collagen Synthesis

The enhancement effect of the composition including the amino acid complex, the calcium pantothenic acid salt, and the mineral component on collagen synthesis was confirmed.

In detail, human normal fibroblasts (available from Korean Cell Line Bank) were inoculated into a 96-well microplate to have a concentration of 2×10³ cells in each well, and cultured in DMEM at 37° C. for 24 hours. Subsequently, the cells of experimental groups in which the prepared composition was included and a medium was replaced with fresh serum-free DMEM were further cultured for 48 hours, whereas the cells of a control group was further cultured for 48 hours without medium replacement. After the culture, the supernatant of each well was collected to measure the amount of procollagen type I C-peptide (PICP) by using a kit (manufactured by Takara, Japan), so as to measure newly synthesized collagen. The measured amount was converted to ng/2×10⁴ cells, and the results are shown in Table 4.

TABLE 4

| Entry | Collagen synthesis amount (ng/2×10⁴) |
|---|---|
| Example 1 | 301 |
| Example 2 | 250 |
| Example 3 | 254 |
| Comparative Example 1 | 201 |
| Comparative Example 2 | 198 |
| Comparative Example 3 | 196 |
| Comparative Example 4 | 131 |
| Comparative Example 5 | 96 |
| Comparative Example 6 | 145 |

As shown in Table 4, the composition including the amino acid complex showed a very high synthesis amount of collagen. In addition, it was confirmed that, as in cases of Examples 1 to 3, the cosmetic compositions that essentially included glycine, L-alanine, L-proline, L-valine, L-leucine, and L-lysine exhibited better effects on the collagen synthesis, whereas Comparative Examples 1 to 3 in which some of these amino acids were not included exhibited somewhat less effects. Meanwhile, as in case of Comparative Example 4, the effect was reduced when the amount of the amino acid complex was low. In addition, in case of Comparative Example 5 in which the amino acid complex was not included, the effect on the collagen synthesis appeared to be low, and in case of Comparative Example 6 in which the amino acid complex was not used together with the mineral component, the effect on the collagen synthesis was also low.

### Experimental Example 3: Confirmation of Cytotoxicity

The cytotoxicity of the composition of Example 1 including the amino acid complex, the calcium pantothenic acid salt, and the mineral component was confirmed.

In detail, the human dermal fibroblasts (CCD-1064Sk, ATCC® CRL-2076™) were subjected to toxicity evaluation by using Iscove’s modified Dulbecco’s medium (IMDM, Welgene, Korea). The human dermal fibroblasts were inoculated into a 24-well plate, cultured for 24 hours, and then replaced with fresh serum-free IMDM. The composition of Example 1 was treated at different concentrations and cultured for 24 hours. After the culture was completed, the medium was replaced with a 10-fold diluted medium with a 2.5 mg/ml MTT solution, and the cells were allowed for a reaction for 4 hours. Then, the supernatant was removed, and 1 ml of DMSO was added to dissolve the MTT-formazan crystals produced, so as to measure absorbance at 570 nm by using an ELISA reader.

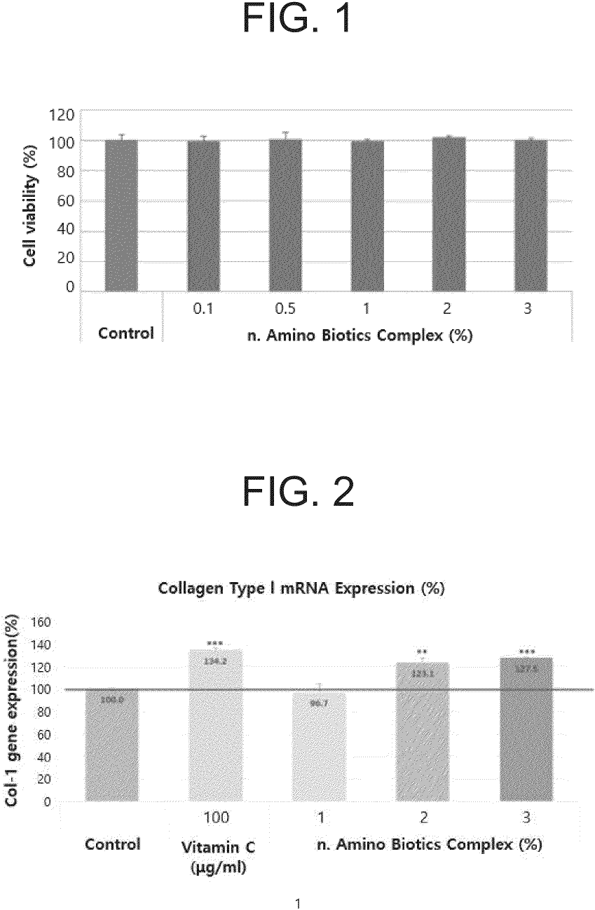

As a result, it was confirmed that the composition of Example 1 had no cytotoxicity as shown in Table 5 and FIG. 1.

TABLE 5

| Sample | Concentration | Mean | Standard deviation | p-value |
|---|---|---|---|---|
| Control group | - | 100.00 | 3.76 | 1.000 |
| Example 1 (%) | 0.1 | 99.32 | 3.10 | 0.821 |
| | 0.5 | 100.68 | 4.02 | 0.841 |
| | 1 | 99.44 | 0.93 | 0.815 |
| | 2 | 101.73 | 1.18 | 0.490 |
| | 3 | 99.91 | 1.08 | 0.970 |

### Experimental Example 4: Confirmation of Gene Expression of Collagen Type I

The effect of the composition of Example 1 including the amino acid complex, the calcium pantothenic acid salt, and the mineral component on gene expression of collagen type I was confirmed.

In detail, vitamin C was used as a positive control group, and the composition of Example 1 was treated at different concentrations, so as to confirm the gene expression level of collagen type I. Human dermal fibroblasts (CCD-1064Sk, ATCC® CRL-2076™) were inoculated into a 60 mm dish using IMDM (Welgene, Korea), cultured for 24 hours, and then replaced with fresh serum-free IMDM. The composition of Example 1 was treated at different concentrations and cultured for 24 hours at 37° C. in a 5 % CO₂ incubator. After removal of the medium, the cells were collected by using QIAzol™ Lysis Reagent (Qiagen), and RNA thereof was isolated according to the protocol of the manufacturer. After quantifying the isolated RNA, cDNA was synthesized with 1 µg of RNA, followed by Real-time PCR (Applied Biosystems, 7500 FAST Real-Time PCR System).

As a result, as shown in Table 6 and FIG. 2, it was confirmed that the composition of Example 1 increased the gene expression of collagen type I to a similar gene expression level with the positive control group.

TABLE 6

| Sample | Concentration | Mean | Standard deviation | p-value |
|---|---|---|---|---|
| Control group | - | 100.0 | 0.00 | 1.000 |
| Vitamin C (µg/mℓ) | 100 | 134.2 | 1.97 | 0.000 |
| Example 1 (%) | 1 | 96.7 | 7.41 | 0.484 |
| | 2 | 123.1 | 4.32 | 0.001 |
| | 3 | 127.5 | 0.41 | 0.000 |

### Experimental Example 5: Confirmation of Gene Expression of Collagen type III

The effect of the composition of Example 1 including the amino acid complex, the calcium pantothenic acid salt, and the mineral component on gene expression of collagen type III was confirmed.

In detail, vitamin C was used as a positive control group, and the composition of Example 1 was treated at different concentrations, so as to confirm the gene expression level of collagen type III. Human dermal fibroblasts (CCD-1064Sk, ATCC® CRL-2076™) were inoculated into a 60 mm dish using IMDM (Welgene, Korea), cultured for 24 hours, and then replaced with fresh serum-free IMDM. The composition of Example 1 was treated at different concentrations and cultured for 24 hours at 37° C. in a 5 % CO₂ incubator. After removal of the medium, the cells were collected by using QIAzol™ Lysis Reagent (Qiagen), and RNA thereof was isolated according to the protocol of the manufacturer. After quantifying the isolated RNA, cDNA was synthesized with 1 µg of RNA, followed by Real-time PCR (Applied Biosystems, 7500 FAST Real-Time PCR System).

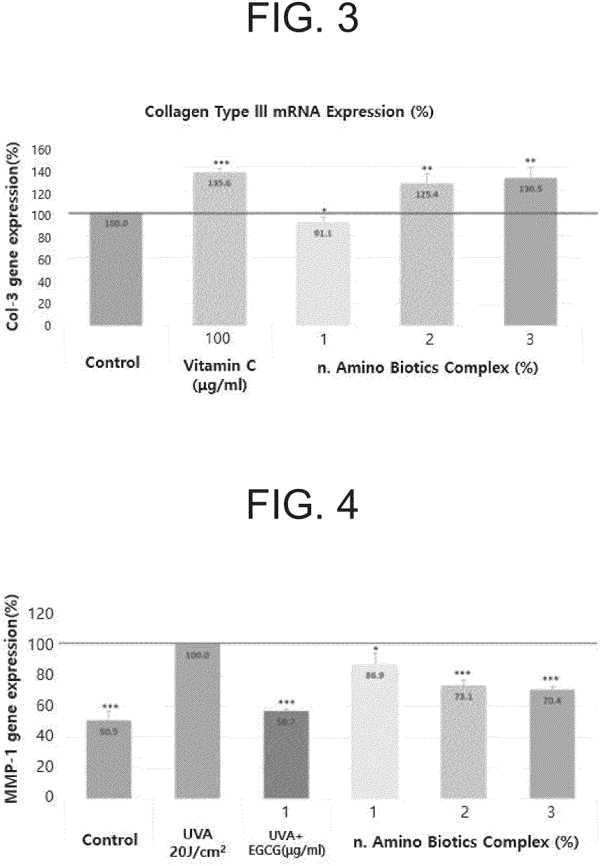

As a result, as shown in Table 7 and FIG. 3, it was confirmed that the composition of Example 1 increased the gene expression of collagen type III to a similar gene expression level with the positive control group.

TABLE 7

| Sample | Concentration | Mean | Standard deviation | p-value |
|---|---|---|---|---|
| Control group | - | 100.0 | 0.00 | 1.000 |
| Vitamin C (µg/mℓ) | 100 | 135.6 | 2.68 | 0.000 |
| [symbol] 1(%) | 1 | 91.1 | 4.17 | 0.021 |
| | 2 | 125.4 | 8.17 | 0.006 |
| | 3 | 130.5 | 8.62 | 0.004 |

### Experimental Example 6: Confirmation of Inhibition of MMP-1 Gene expression

The inhibitory effect of the composition of Example 1 including the amino acid complex, the calcium pantothenic acid salt, and the mineral component on gene expression of MMP-1 was confirmed.

In detail, as a positive control group, cells damaged by ultraviolet A (UVA) irradiation were treated with EGCG, and the composition of Example 1 was treated at different concentrations, so as to confirm the gene expression level of MMP-1. Human dermal fibroblasts (CCD-1064Sk, ATCC® CRL-2076™) were inoculated into a 60-mm dish using IMDM (Welgene, Korea) and cultured for 24 hours, followed by UVA irradiation. Afterwards, the medium was replaced with fresh serum-free IMDM, and samples were each treated at different concentrations and cultured for 24 hours at 37° C. in a 5 % CO₂ incubator. After removal of the medium, the cells were collected by using QIAzol™ Lysis Reagent (Qiagen), and RNA thereof was isolated according to the protocol of the manufacturer. After quantifying the isolated RNA, cDNA was synthesized with 1 µg of RNA, followed by Real-time PCR (Applied Biosystems, 7500 FAST Real-Time PCR System).

As a result, as shown in Table 8 and FIG. 4, it was confirmed that the composition of Example 1 reduced the gene expression of MMP-1 in a concentration-dependent manner.

TABLE 8

| Sample | Concentration | Mean | Standard deviation | p-value |
|---|---|---|---|---|
| Control group | - | 50.5 | 6.13 | 0.000 |
| UVA | - | 100.0 | 0.00 | 1.000 |
| UVA+EGCG (µg/mℓ) | 1 | 56.7 | 1.27 | 0.000 |
| [symbol] 1(%) | 1 | 86.9 | 7.10 | 0.033 |
| | 2 | 73.1 | 3.30 | 0.000 |
| | 3 | 70.4 | 1.94 | 0.000 |

### Preparation Examples: Preparation of Cosmetic Composition

In order to conduct a clinical test for effects of a cosmetics including all of the amino acid complex, the calcium pantothenate, and the mineral component on the amelioration of skin wrinkles and improvement of elasticity, cosmetic compositions were prepared with the components and amounts as shown in Table 9.

A cosmetic composition not including the amino acid complex was prepared in Comparative Preparation Example 1. Specific components and amounts are as shown in Table 9.

TABLE 9

| Component | Amount (wt%) | | | |
|---|---|---|---|---|
| | Preparation Example 1 | Preparation Example 2 | Preparation Example 3 | Comparative Preparation Example 1 |
| Vaseline | 7.0 | 7.0 | 7.0 | 7.0 |
| Liquid paraffin | 5.0 | 5.0 | 5.0 | 5.0 |
| Wax | 2.0 | 2.0 | 2.0 | 2.0 |
| Polysorbate-60 | 2.0 | 2.0 | 2.0 | 2.0 |
| Sorbitan sesquioleate | 2.5 | 2.5 | 2.5 | 2.5 |
| Squalane | 3.0 | 3.0 | 3.0 | 3.0 |
| Propylene glycol | 6.0 | 6.0 | 6.0 | 6.0 |
| Glycerin | 4.0 | 4.0 | 4.0 | 4.0 |
| Triethanolamine | 0.5 | 0.5 | 0.5 | 0.5 |
| Carboxyvinyl polymer | 0.5 | 0.5 | 0.5 | 0.5 |
| Tocopherol acetate | 0.1 | 0.1 | 0.1 | 0.1 |
| Purified water | To 100 | To 100 | To 100 | To 100 |
| Calcium pantothenate | 0.7 | 0.7 | 0.7 | 0.7 |
| Mineral component^{a} | 0.6 | 0.6 | 0.6 | 0.6 |
| Amino acid complex A | 25.0 | - | - | - |
| Amino acid complex B | - | 25.0 | - | - |
| Amino acid complex F | - | - | 25.0 | - |
| Fragrance, preservative | Trace amount | Trace amount | Trace amount | Trace amount |

Mineral component^{a}: Complex of Zn-gluconate, Mg-gluconate, and Ca-gluconate at a weight ratio of 1:1:1

### Experimental Example 7: Confirmation of Effect of Skin Wrinkle Amelioration

In order to examine effects of the cosmetic compositions of Preparation Examples 1 to 3 and Comparative Preparation Example 1 on the amelioration of skin wrinkles, skin wrinkles were measured from 40 healthy women aged between 35 to 50 as follows.

First, the subject women were divided into four groups, and Group A (10 people), Group B (10 people), Group C (10 people), and Group D (10 people) were treated with the formulations of Preparation Examples 1 to 3 and Comparative Preparation Example 1, respectively, by applying the formulations onto the face twice a day (in the morning and night) for 3 months. After 3 months, the degree of amelioration of wrinkles was evaluated through a questionnaire of the subject women and image analysis on the wrinkles.

The questionnaire of the subject women was determined as 4 stages of no improvement, slight improvement, moderate improvement, and significant improvement compared to the condition before use, and the results are shown in Table 10.

For the evaluation of the wrinkles by image analysis, a replica was harvested from an area underneath the eye before the experiments began, and a replica immediately after the end of the experiment was harvested from the same area underneath the eye, so as to measure density of the wrinkles by two-dimensional analysis of the wrinkles through image analysis. The measurement results of the wrinkle density by image analysis were indicated by the reduction rate of the wrinkle density before use, and the results are shown in Table 11.

TABLE 10

| Sample | No improvement (people) | Slight improvement (people) | Moderate improvement (people) | Significant improvement (people) |
|---|---|---|---|---|
| Preparation Example 1 | 0 | 1 | 2 | 7 |
| Preparation Example 2 | 0 | 2 | 3 | 5 |
| Preparation Example 3 | 2 | 1 | 3 | 4 |
| Comparative Preparation Example 1 | 4 | 4 | 1 | 1 |

TABLE 11

| Sample | Wrinkle density reduction rate |
|---|---|
| Preparation Example 1 | 55 |
| Preparation Example 2 | 49 |
| Preparation Example 3 | 43 |
| Comparative Preparation Example 1 | 15 |

As shown in the results of Tables 10 and 11, it was found that the cosmetic composition including the amino acid complex exhibited very excellent effects on the amelioration of skin wrinkles compared to the composition not including the amino acid complex.

### Experimental Example 8: Confirmation of Effect on Skin Elasticity improvement

In order to examine the enhancement effects of the cosmetic compositions of Preparation Examples 1 to 3 and Comparative Preparation Example 1 on skin elasticity, measurements were carried out as follows.

First, 40 healthy women over 20 years old (average age 37) were divided into four groups, and Group A (10 people), Group B (10 people), Group C (10 people), and Group D (10 people) were treated with the formulations of Preparation Examples 1 to 3 and Comparative Preparation Example 1, respectively, by applying the formulations around the eyes twice a day (in the morning and night) for 12 weeks under the conditions of a temperature in a range of about 24° C. to about 26° C. and a humidity of 75 %. Then, the skin elasticity was measured by using a skin elasticity measuring device (Cutometer MPA580, Conrage+Khazaka company, Germany). The test results were calculated as R8 value (R8 (at 12 weeks)-R8 (at 0 week) of the cutometer MPA580, and the results are shown in Table 12. Here, the R8 value represents the property of skin viscoelasticity.

TABLE 12

| Sample | Skin elasticity effect |
|---|---|
| Preparation Example 1 | 0.79 |
| Preparation Example 2 | 0.69 |
| Preparation Example 3 | 0.51 |
| Comparative Preparation Example 1 | 0.29 |

As shown in the results of Table 12, it was confirmed that the cosmetic composition including the amino acid complex significantly increased the enhancement effect on skin elasticity compared to the composition not including the amino acid complex.

The composition according to an aspect including the amino acid complex as an active ingredient may proliferate cells, promote expression and synthesis of collagen, and have an effect on skin wrinkles and skin elasticity, and thus may be effectively used for overall skin improvements.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A cosmetic composition for skin improvement, comprising, as an active ingredient, an amino acid complex comprising glycine, L-alanine, L-proline, L-valine, L-leucine, L-lysine, serine, glutamic acid, aspartic acid, arginine, and histidine.

2. The cosmetic composition of claim 1, further comprising, as active ingredients, calcium pantothenate and a mineral component.

3. The cosmetic composition of claim 2, wherein the mineral component comprises, in a form of a single substance or a salt, at least three selected from the group consisting of calcium (Ca), magnesium (Mg), copper (Cu), and zinc (Zn).

4. The cosmetic composition of claim 3, wherein the mineral in a form of a salt comprises aspartate or gluconate of the mineral component.

5. The cosmetic composition of claim 1, wherein the cosmetic composition comprises, based on a total weight of the amino acid complex, glycine in an amount of about 20.0 wt% to about 35.0 wt%, L-alanine in an amount of about 15.0 wt% to about 30.0 wt%, L-proline in an amount of about 15.0 wt% to about 20.0 wt%, L-valine in an amount of about 10.0 wt% to about 25.0 wt%, L-leucine in an amount of about 1.0 wt% to about 5.0 wt%, L-lysine in an amount of about 1.0 wt% to about 5.0 wt%, serine in an amount of about 5.0 wt% to about 10.0 wt%, glutamic acid in an amount of about 5.0 wt% to about 10.0 wt%, aspartic acid in an amount of about 1.0 wt% to about 5.0 wt%, arginine in an amount of about 1.0 wt% to about 5.0 wt%, and histidine in an amount of about 1.0 wt% to about 5.0 wt%.

6. The cosmetic composition of claim 1, wherein the cosmetic composition comprises the amino acid complex in an amount of about 10.0 wt% to about 40.0 wt% based on a total weight of the cosmetic composition.

7. The cosmetic composition of claim 2, wherein the cosmetic composition comprises calcium pantothenate in an amount of about 0.1 wt% to about 5.0 wt% based on a total weight of the cosmetic composition.

8. The cosmetic composition of claim 2, wherein the cosmetic composition comprises the mineral component in an amount of about 0.1 wt% to about 5.0 wt% based on a total weight of the cosmetic composition.

9. The cosmetic composition of claim 1, wherein the skin improvement is at least one selected from the group consisting of improvement of skin barrier, prevention or improvement of skin damage, skin moisturizing, amelioration of skin wrinkles, inhibition of skin aging, improvement of skin elasticity, skin soothing, and skin regeneration.

10. A food composition for skin improvement, comprising, as an active ingredient, an amino acid complex comprising glycine, L-alanine, L-proline, L-valine, L-leucine, L-lysine, serine, glutamic acid, aspartic acid, arginine, and histidine.

11. The food composition of claim 10, further comprising, as active ingredients, calcium pantothenate and a mineral component.
